(19) United States
(12) Patent Application Publication
(10) Pub. No.: US 2011/0232763 A1
(43) Pub. Date: Sep. 29, 2011

(54) SPIROBIFLUORENE-BASED COMPOUND AND DYE-SENSITIZED SOLAR CELL USING THE SAME

(75) Inventors: Moon-Sung KANG, Yongin-si (KR); Ji-Won Lee, Yongin-si (KR); Byong-Cheol Shin, Yongin-si (KR); Jae-Jung Ko, Yongin-si (KR); Hyun-Bong Choi, Yongin-si (KR)

(73) Assignee: Samsung SDI Co., Ltd., Yongin-si (KR)

(21) Appl. No.: 12/904,992

(22) Filed: Oct. 14, 2010

(30) Foreign Application Priority Data
Mar. 24, 2010 (KR) 10-2010-0026399

(51) Int. Cl. H01L 31/0256; C07D 409/14; C07D 495/04; C07C 63/00; C07D 307/26

(52) U.S. Cl. 136/263; 549/43; 549/50; 562/405; 549/429

(57) ABSTRACT

A spirobifluorene-based compound and a dye-sensitized solar cell using the spirobifluorene-based compound as a dye.

### CROSS-REFERENCE TO RELATED APPLICATIONS

This application claims the benefit of Korean Patent Application No. 10-2010-0026399, filed on Mar. 24, 2010, in the Korean Intellectual Property Office, the disclosure of which is incorporated herein in its entirety by reference.

### BACKGROUND

1. Field

One or more embodiments relate to a spirobifluorene-based compound and a dye-sensitized solar cell using the same.

2. Description of the Related Technology

Unlike silicon solar cells, dye-sensitized solar cells are photoelectrochemical solar cells that are composed of photosensitive dye molecules, as main constituents, that may produce electron-hole pairs by absorbing visible rays, and a transition metal oxide that transfers the produced electrons. Dye-sensitized solar cells may be manufactured at lower cost than silicon-based solar cells, and since they use transparent electrodes, the cells may be applied to external glass walls of a building or glass greenhouse. However, in the past, dye-sensitized solar cells have had limited practical application due to their low photoelectric conversion efficiency.

The photoelectric conversion efficiency of a dye-sensitized solar cell is proportional to the quantity of electrons produced from the absorption of solar rays. Thus, to increase the photoelectric conversion efficiency, the quantity of the produced electrons may be increased by absorbing more sunlight or by increasing the amount of dye adsorbed, or the excited electrons so produced may be prevented from being used to cause electron-hole recombination.

To increase the adsorption amount of dye per unit area, oxide semiconductor particles need to be nano-sized, and, to increase the absorption of the sunlight, the reflectivity of a platinum electrode may be increased, or a micro-sized oxide semiconductor light scattering agent should be included to increase the absorption of solar rays. However, these conventional methods have limitations in terms of increasing the photoelectric conversion efficiency of dye-sensitized solar cells. Therefore, there is an urgent need to develop a novel method of improving the photoelectric conversion efficiency of the dye-sensitized solar cells.

### SUMMARY

One or more embodiments include a spirobifluorene-based compound having excellent thermal stability and a dye-sensitized solar cell using the same, whereby the photoelectric conversion efficiency is enhanced.

Additional aspects will be set forth in part in the description which follows and, in part, will be apparent from the description, or may be learned by practice of the presented embodiments.

According to one or more embodiments, a spirobifluorene-based compound represented by Formula 1 below is provided.

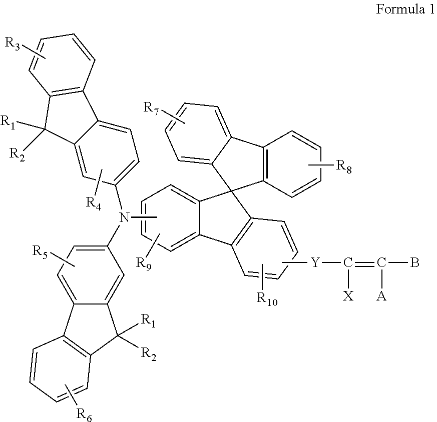

wherein Y denotes a chemical bond, or a substituted or unsubstituted C₆-C₃₀ arylene group, a substituted or unsubstituted C₂-C₃₀ heteroarylene group, or a substituted or unsubstituted C₂-C₃₀ alkynylene group,

X is hydrogen, a substituted or unsubstituted C₁-C₃₀ alkyl group, a substituted or unsubstituted C₁-C₃₀ alkoxy group, a substituted or unsubstituted C₆-C₃₀ aryl group, a substituted or unsubstituted C₂-C₃₀ heteroaryl group, or a cyano group,

R₁ and R₂ are each independently hydrogen, a substituted or unsubstituted C₁-C₃₀ alkyl group, a substituted or unsubstituted C₁-C₃₀ alkoxy group, a substituted or unsubstituted C₆-C₃₀ aryl group, or a substituted or unsubstituted C₂-C₃₀ heteroaryl group,

A is a cyano group or a carboxyl group,

B is an acidic functional group, and

R₃ through R₁₀ are monosubstituted or multi-substituted groups, and are each independently hydrogen, a substituted or unsubstituted C₁-C₃₀ alkyl group, a substituted or unsubstituted C₁-C₃₀ alkoxy group, a substituted or unsubstituted C₆-C₃₀ aryl group, a substituted or unsubstituted C₂-C₃₀ heteroaryl group, a substituted or unsubstituted C₂-C₃₀ alkynyl group, a substituted or unsubstituted C₃-C₃₀ carbocyclic group, a substituted or unsubstituted C₂-C₃₀ heterocyclic group, a halogen atom, a hydroxyl group, a cyano group, a thiol group, or an amino group.

According to one or more embodiments, a dye-sensitized solar cell includes a first electrode, a light absorption layer formed on a surface of the first electrode, a second electrode disposed to face the first electrode on which the light absorption layer is formed, and an electrolyte disposed between the first electrode and the second electrode; and a spirobifluorene-based compound according to any one of claims 1 through 8, wherein the spirobifluorene-based compound is used as a dye.

### BRIEF DESCRIPTION OF THE DRAWINGS

These and/or other aspects will become apparent and more readily appreciated from the following description of the embodiments, taken in conjunction with the accompanying drawings of which:

FIG. 1 is a cross-sectional view illustrating a structure of a dye-sensitized solar cell according to an embodiment;

FIG. 2 is a graph showing variation in incident photon to current efficiency (IPCE) with respect to unit wavelength of dye-sensitized solar cells manufactured according to Preparation Examples 1 through 3; and

FIG. 3 is a graph showing UV-photoluminescence (PL) characteristics of compounds represented by Formulae 4 to 6, prepared according to Synthesis Examples 1 through 3.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description.

According to an embodiment, there is provided a spirobifluorene-based compound represented by Formula 1 below:

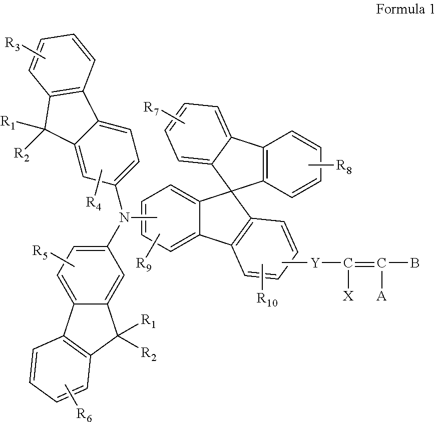

wherein Y denotes a chemical bond, or a substituted or unsubstituted C₆-C₃₀ arylene group, a substituted or unsubstituted C₂-C₃₀ heteroarylene group, or a substituted or unsubstituted C₂-C₃₀ alkynylene group,

X is hydrogen, a substituted or unsubstituted C₁-C₃₀ alkyl group, a substituted or unsubstituted C₁-C₃₀ alkoxy group, a substituted or unsubstituted C₆-C₃₀ aryl group, a substituted or unsubstituted C₂-C₃₀ heteroaryl group, or a cyano group,

R₁ and R₂ are each independently hydrogen, a substituted or unsubstituted C₁-C₃₀ alkyl group, a substituted or unsubstituted C₁-C₃₀ alkoxy group, a substituted or unsubstituted C₆-C₃₀ aryl group, or a substituted or unsubstituted C₂-C₃₀ heteroaryl group,

A is a cyano group or a carboxyl group,

B is an acidic functional group, and

R₃ through R₁₀ are monosubstituted or multi-substituted groups, and are each independently hydrogen, a substituted or unsubstituted C₁-C₃₀ alkyl group, a substituted or unsubstituted C₁-C₃₀ alkoxy group, a substituted or unsubstituted C₆-C₃₀ aryl group, a substituted or unsubstituted C₂-C₃₀ heteroaryl group, a substituted or unsubstituted C₂-C₃₀ alkynyl group, a substituted or unsubstituted C₃-C₃₀ carbocyclic group, a substituted or unsubstituted C₂-C₃₀ heterocyclic group, a halogen atom, a hydroxyl group, a cyano group, a thiol group, or an amino group.

The spirobifluorene-based compound of Formula 1 may be a compound represented by Formula 2 below:

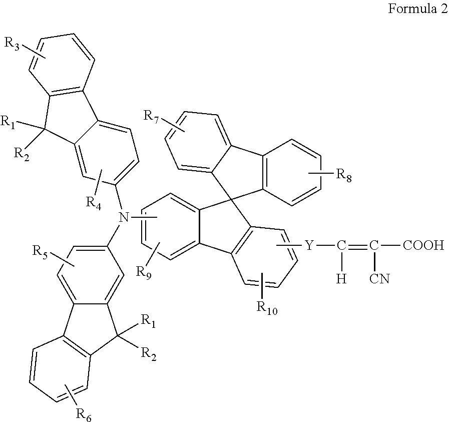

wherein Y denotes a chemical bond, or a substituted or unsubstituted C₆-C₃₀ arylene group, a substituted or unsubstituted C₂-C₃₀ heteroarylene group, or a substituted or unsubstituted C₂-C₃₀ alkynylene group,

R₁ and R₂ are each independently hydrogen, a substituted or unsubstituted C₁-C₃₀ alkyl group, a substituted or unsubstituted C₁-C₃₀ alkoxy group, a substituted or unsubstituted C₆-C₃₀ aryl group, OR a substituted or unsubstituted C₂-C₃₀ heteroaryl group, and

R₃ through R₁₀ are mono-substituted or multi-substituted groups, and are each independently hydrogen, a substituted or unsubstituted C₁-C₃₀ alkyl group, a substituted or unsubstituted C₁-C₃₀ alkoxy group, a substituted or unsubstituted C₆-C₃₀ aryl group, a substituted or unsubstituted C₂-C₃₀ heteroaryl group, a substituted or unsubstituted C₂-C₃₀ alkynyl group, a substituted or unsubstituted C₃-C₃₀ carbocyclic group, a substituted or unsubstituted C₂-C₃₀ heterocyclic group, a halogen atom, a hydroxyl group, a cyano group, a thiol group, or an amino group.

The spirobifluorene-based compound of Formula 1 includes spirobifluorene having excellent thermal stability as a spacer, while not wishing to be bound to a particular theory this allows the compound to absorb more light with long wavelengths. The spirobifluorene activates charge separation between an electron donor ligand and an electron acceptor ligand and prevents overlapping between molecules, whereby a dye-sensitized solar cell using the compound of Formula 1 has a high open-circuit voltage. For example, the open-circuit voltage is from about 0.6 to about 1.0 V.

The spirobifluorene-based compound of Formula 1 may be a compound represented by Formula 3 below:

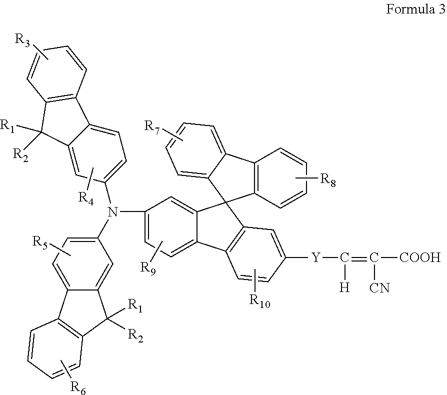

wherein Y denotes a chemical bond, or a substituted or unsubstituted C₆-C₃₀ arylene group, a substituted or unsubstituted C₂-C₃₀ heteroarylene group, or a substituted or unsubstituted C₂-C₃₀ alkynylene group,

R₁ and R₂ are each independently hydrogen, a substituted or unsubstituted C₁-C₃₀ alkyl group, a substituted or unsubstituted C₁-C₃₀ alkoxy group, a substituted or unsubstituted C₆-C₃₀ aryl group, or a substituted or unsubstituted C₂-C₃₀ heteroaryl group, and

R₃ through R₁₀ are mono-substituted or multi-substituted groups, and are each independently hydrogen, a substituted or unsubstituted C₁-C₃₀ alkyl group, a substituted or unsubstituted C₁-C₃₀ alkoxy group, a substituted or unsubstituted C₆-C₃₀ aryl group, a substituted or unsubstituted C₂-C₃₀ heteroaryl group, a substituted or unsubstituted C₂-C₃₀ alkynyl group, a substituted or unsubstituted C₃-C₃₀ carbocyclic group, a substituted or unsubstituted C₂-C₃₀ heterocyclic group, a halogen atom, a hydroxyl group, a cyano group, a thiol group, or an amino group.

Y denotes a single bond, a C₆-C₂₀ arylene group, or a C₂-C₂₀ heteroarylene group. For example, Y may be selected from the groups represented by the following formulae:

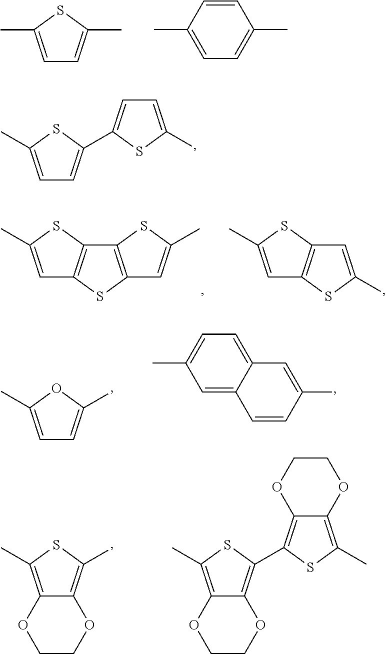

As described above, when Y is a phenylene group or thiophene group, the compound of Formula 1 absorbs more light with long wavelengths.

B of formula 1 is at least one selected from the group consisting of a carboxyl group, a phosphorous group, a sulfonic acid, a phosphinic acid group, an oxycarboxylic acid group, a boric acid group, and a squaric acid group. For example, B may be a carboxyl group (—COOH).

The spirobifluorene-based compound of Formula 1 may be one of the compounds represented by Formulae 4 through 6 below:

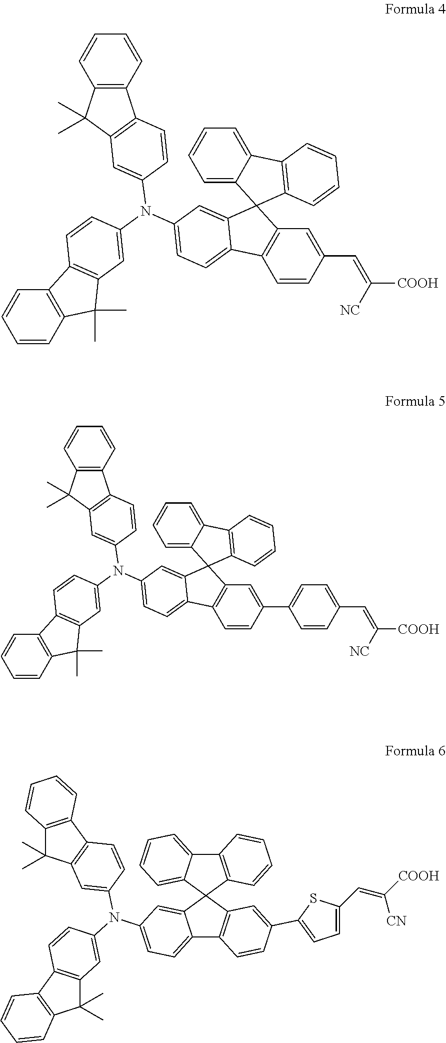

The spirobifluorene-based compound of Formula 1 may be prepared as follows.

The spirobifluorene-based compound of Formula 1 may be synthesized by reacting a compound represented by Formula 7 below and a compound represented by Formula 8 below:

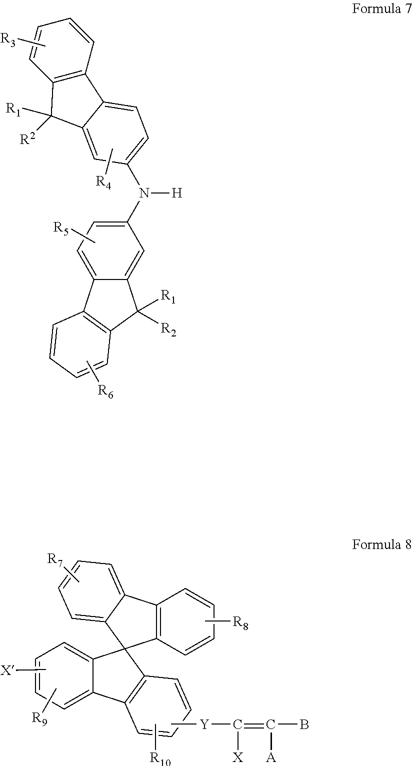

In Formula 8, Y denotes a chemical bond, or a substituted or unsubstituted C₆-C₃₀ arylene group, a substituted or unsubstituted C₂-C₃₀ heteroarylene group, or a substituted or unsubstituted C₂-C₃₀ alkynylene group,

X is hydrogen, a substituted or unsubstituted C₁-C₃₀ alkyl group, a substituted or unsubstituted C₁-C₃₀ alkoxy group, a substituted or unsubstituted C₆-C₃₀ aryl group, a substituted or unsubstituted C₂-C₃₀ heteroaryl group, or a cyano group,

R₁ and R₂ are each independently hydrogen, a substituted or unsubstituted C₁-C₃₀ alkyl group, a substituted or unsubstituted C₁-C₃₀ alkoxy group, a substituted or unsubstituted C₆-C₃₀ aryl group, or a substituted or unsubstituted C₂-C₃₀ heteroaryl group,

A is a cyano group or a carboxyl group,

B is an acidic functional group, and

R₃ through R₁₀ are mono-substituted or multi-substituted groups, and are each independently hydrogen, a substituted or unsubstituted C₁-C₃₀ alkyl group, a substituted or unsubstituted C₁-C₃₀ alkoxy group, a substituted or unsubstituted C₆-C₃₀ aryl group, a substituted or unsubstituted C₂-C₃₀ heteroaryl group, a substituted or unsubstituted C₂-C₃₀ alkynyl group, a substituted or unsubstituted C₃-C₃₀ carbocyclic group, a substituted or unsubstituted C₂-C₃₀ heterocyclic group, a halogen atom, a hydroxyl group, a cyano group, a thiol group, or an amino group,

X is a halogen atom.

The halogen atom may be, for example, iodine (I), fluorine (F), bromine (Br), or chlorine (Cl).

In some embodiments, the reaction between the compound of Formula 7 and the compound of Formula 8 may be performed by adding palladium acetate, tertiary butylphosphine, and cesium carbonate to a mixture of the compound of Formula 7 and the compound of Formula 8, adding toluene as a solvent to the resulting mixture, and then refluxing the resultant mixture.

The spirobifluorene-based compound of Formula 1 may be used as a dye for a dye-sensitized solar cell.

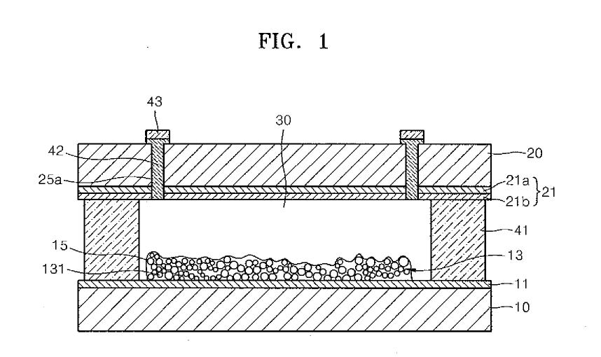

FIG. 1 is a cross-sectional view illustrating a structure of a dye-sensitized solar cell according to an embodiment.

Referring to FIG. 1, the dye-sensitized solar cell according to the present embodiment includes a first substrate 10 on which a first electrode 11, a photoelectrode 13, and a dye 15 are formed, a second substrate 20 on which a second electrode 21 is formed, and an electrolyte 30 disposed between the first electrode 11 and the second electrode 21 such that the first substrate 10 and the second substrate 20 face each other. A case (not shown) may be disposed at an outer side of the first substrate 10 and the second substrate 20. The structure of the dye-sensitized solar cell will now be described in more detail.

In the present embodiment, the first substrate 10, which supports the first electrode 11, may be transparent to allow external light to be incident on the first substrate 10. Thus, the first substrate 10 may be formed of glass or plastic. The plastic may be polyethylene terephthalate (PET), polyethylene naphthalate (PEN), polycarbonate (PC), polypropylene (PP), polyimide (PI), triacetyl cellulose (TAC), or the like.

The first electrode 11 formed on the first substrate 10 may be formed of a transparent material such as ZnO—Ga₂O₃, ZnO-Aₗ₂O₃, at least one selected from indium tin oxide, indium oxide, tin oxide, zinc oxide, sulfur oxide, fluorine oxide, and mixtures thereof, or the like. The first electrode 11 may be in the form of a single film or laminated film formed of the transparent material.

The photoelectrode 13 is formed on the first electrode 11. The photoelectrode 13 includes titanium oxide particles 131, and has an appropriate average pore size, thereby easily transferring the electrolyte 30.

The thickness of the photoelectrode 13 may be from about 10 to 3000 nm, for example, from about 10 to about 1000 nm. However, the present embodiments are not limited thereto, and the thickness of the photoelectrode 13 may vary according to technology advancement, and the like.

The dye 15 that absorbs external light to produce excited electrons is adsorbed onto a surface of the photoelectrode 13. The dye 15 may be the spirobifluorene-based compound of Formula 1. For example, one of the compounds of Formula 4 through 6 may be used as the dye 15.

The second substrate 20 disposed to face the first substrate 10 supports the second electrode 21, and may be transparent. Thus, the second substrate 20 may be formed of glass or plastic, as is the first substrate 10.

The second electrode 21 formed on the second substrate 20 is disposed to face the first electrode 11, and may include a transparent electrode 21a and a catalyst electrode 21b.

The transparent electrode 21a may be formed of a transparent material such as indium tin oxide, fluoro tin oxide, antimony tin oxide, zinc oxide, tin oxide, ZnO—Ga2O3, ZnO—Al2O3, or the like. The transparent electrode 21a may be in the form of a single film or a laminated film formed of the transparent material.

The catalyst electrode 21b activates redox couples, and may be formed be a platinum electrode.

The first substrate 10 and the second substrate 20 are attached to each other using an adhesive 41. The electrolyte 30 is injected into the interior between the first electrode 11 and the second electrode 21 through holes 25a that penetrate the second substrate 20 and the second electrode 21. The electrolyte 30 is uniformly diffused into the photoelectrode 13. The electrolyte 30 receives electrons from the second electrode 21 and transfers the electrons to the dye 15 through reduction and oxidation. The holes 25a penetrating the second substrate 20 and the second electrode 21 are sealed by an adhesive 42 and a cover glass 43.

Although not illustrated in FIG. 1, a metal oxide film, which is a general porous film, may be further formed between the first electrode 11 and the photoelectrode 13. In this regard, the photoelectrode 13 acts as a light scattering electrode and is capable of adsorbing a large amount of dye, thereby addressing the disadvantages of conventional light scattering electrodes. Accordingly, the dye-sensitized solar cell may have high efficiency.

The porous film may be formed of metal oxide particles, and examples of the metal oxide may include titanium oxide, zinc oxide, tin oxide, strontium oxide, indium oxide, iridium oxide, lanthan oxide, vanadium oxide, molybdenum oxide, tungsten oxide, niobium oxide, magnesium oxide, aluminum oxide, yttrium oxide, scandium oxide, samarium oxide, gallium oxide, and strontium titanium oxide. The metal oxide particles may be TiO₂ particles, SnO₂ particles, WO₃ particles, ZnO particles, or complexes thereof.

The substituents in formulae 1 through 8 are defined as follows.

The alkyl group used herein is in a linear or branched form, and may be methyl, ethyl, propyl, iso-butyl, sec-butyl, pentyl, iso-amyl, hexyl, heptyl, octyl, nonanyl, dodecyl, or the like. At least one hydrogen atom of the alkyl group may be substituted with a deuterium atom, a halogen atom, a hydroxyl group, a nitro group, a cyano group, an amino group, an amidino group, hydrazine, hydrazone, a carboxyl group or salts thereof, a sulfonic acid group or salts thereof, a phosphoric acid group or salts thereof, a C₁-C₁₀ alkyl group, a C₁-C₁₀ alkoxy group, a C₂-C₁₀ alkenyl group, a C₂-C₁₀ alkynyl group, a C₆-C₁₆ aryl group, or a C₄-C₁₆ heteroaryl group.

The alkoxy group used herein may be a group represented by —OA where A is an unsubstituted C₁-C₅₀ alkyl group, and may be methoxy, ethoxy, propoxy, isopropyloxy, butoxy, penthoxy, or the like. At least one hydrogen atom of the alkoxy group may be substituted with the same substituent as in alkyl group described above.

The aryl group used herein is used alone or in combination, and refers to a carbocyclic aromatic system containing at least one ring, wherein the rings can be attached to each other using a pedant method or fused with each other. The term “aryl” refers to an aromatic radical, including phenyl, naphthyl, tetrahydronaphthyl, or the like. At least one hydrogen atom of the aryl group may be substituted with the same substituent as in alkyl group described above.

The heteroaryl group used herein refers to an aromatic organic compound which contains at least one heteroatom selected from the group consisting of nitrogen (N), oxygen (O), phosphorus (P), and sulfur (S). At least one hydrogen atom of the heteroaryl group may be replaced with the same substituent as in the alkyl group described above.

The heterocyclic group used herein refers to a ring group containing a heteroatom such as N, S, P, or O. At least one hydrogen atom of the heterocyclic group may be replaced with the same substituent as in the alkyl group described above.

The carbocyclic group used herein refers to a cyclic alkyl group. At least one hydrogen atom of the carbocyclic group may be replaced with the same substituent as in the alkyl group described above.

The alkylene group used herein may be methylene, ethylene, or the like. At least one hydrogen atom of the alkylene group may be replaced with the same substituent as in the alkyl group described above.

At least one hydrogen atom of the alkenylene group and the alkynylene group may be replaced with the same substituent as in the alkyl group described above.

The arylene group used herein may be phenylene, biphenylene, or the like, and at least one hydrogen atom of the arylene group may be replaced with the same substituent as in the alkyl group described above.

At least one hydrogen atom of the heteroarylene group may be replaced with the same substituent as in the alkyl group described above.

Each of the heteroaryloxy group, the arylalkyl group, the aryloxy group, the carbocyclic alkyl group, the heterocyclic alkyl group, and the heteroarylalkyl group may be replaced with the same substituent as in the alkyl group described above.

One or more embodiments will now be described in further detail with reference to the following examples. However, these examples are for illustrative purposes only and are not intended to limit the scope of the present embodiments.

The compound of Formula 4 is synthesized according to Synthesis Example 1 through Reaction Scheme 1 below, and the compound of Formula 5 and the compound of Formula 6 are synthesized according to Synthesis Example 2 and Synthesis Example 3, respectively, through Reaction Scheme 2 below:

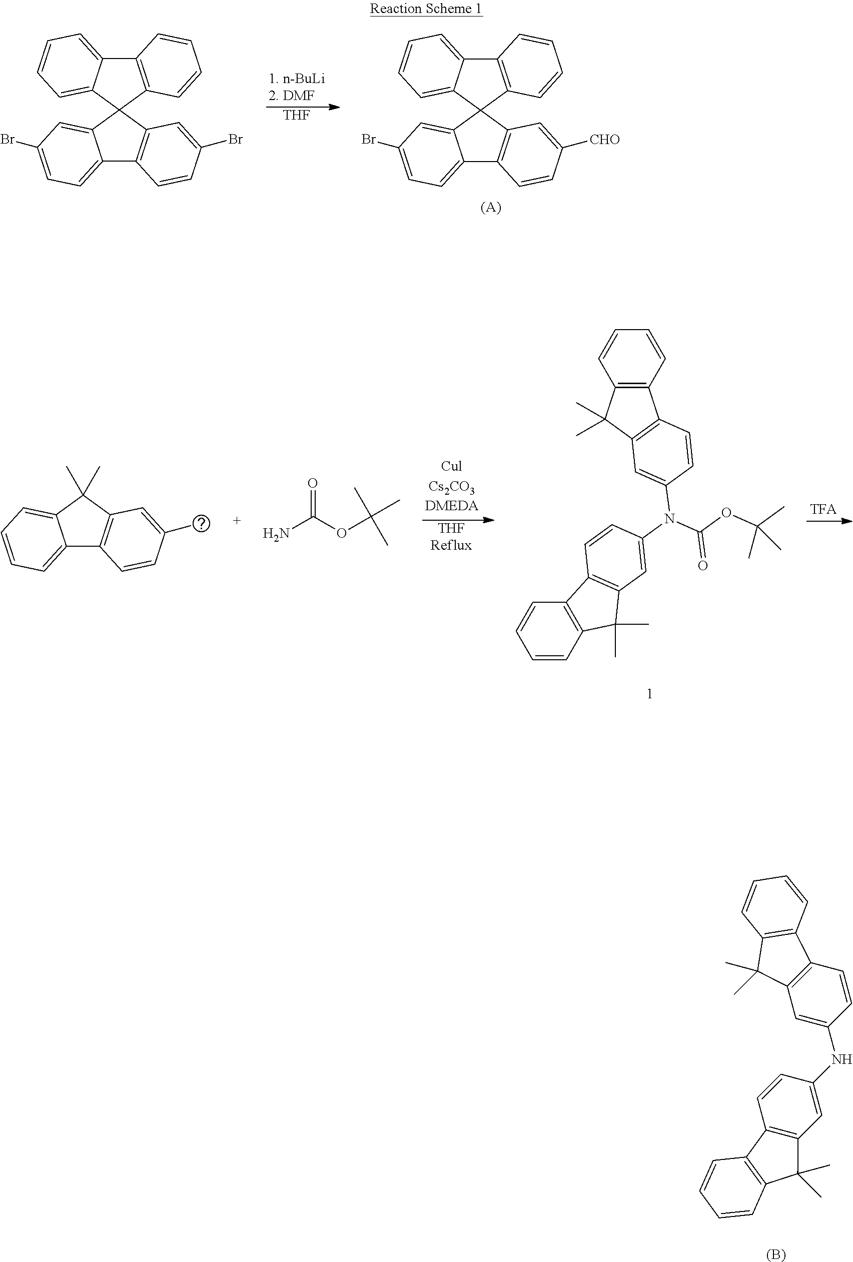

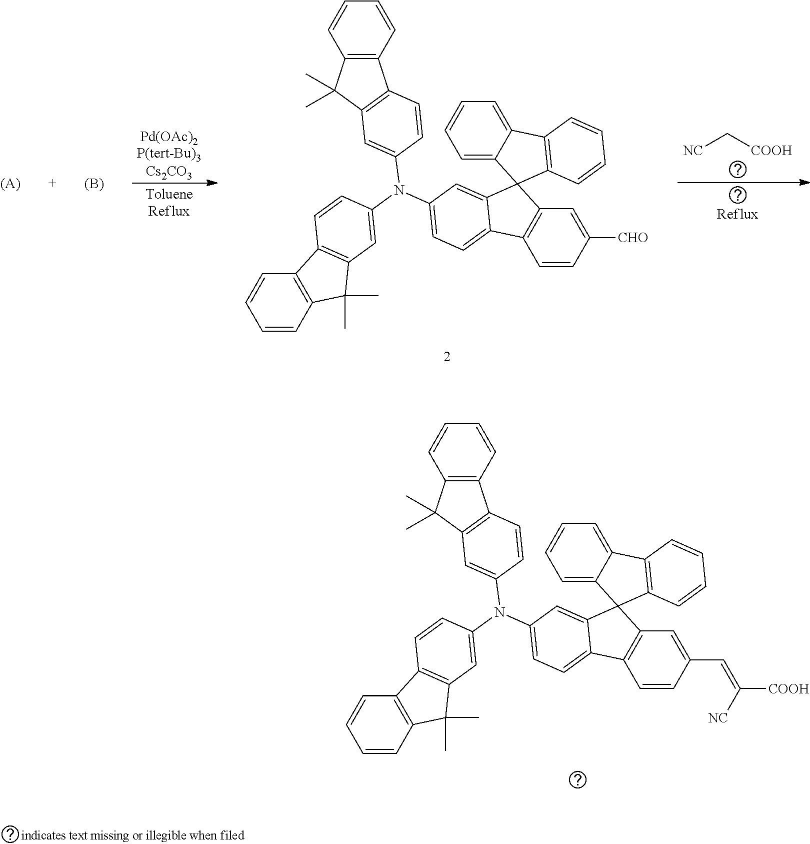

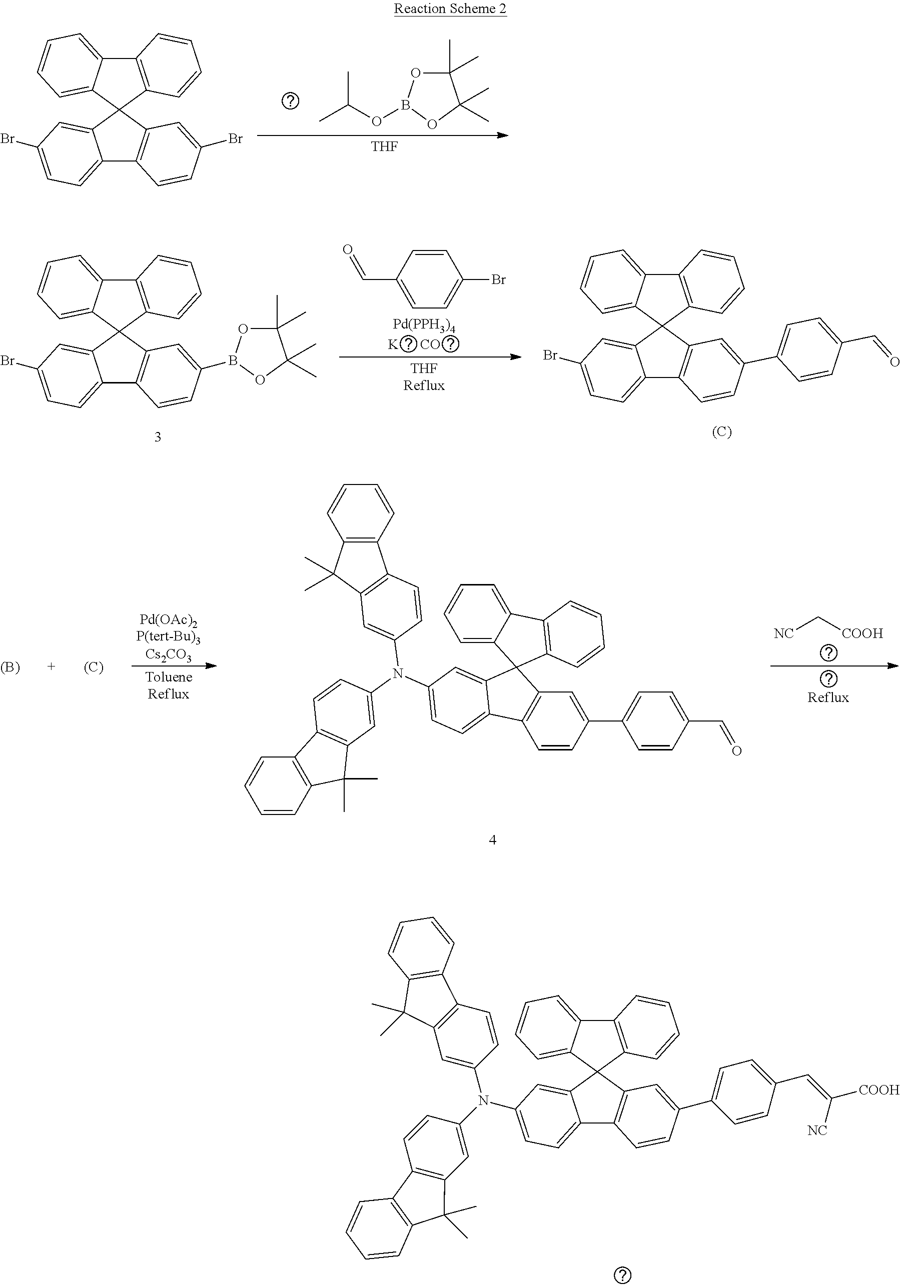

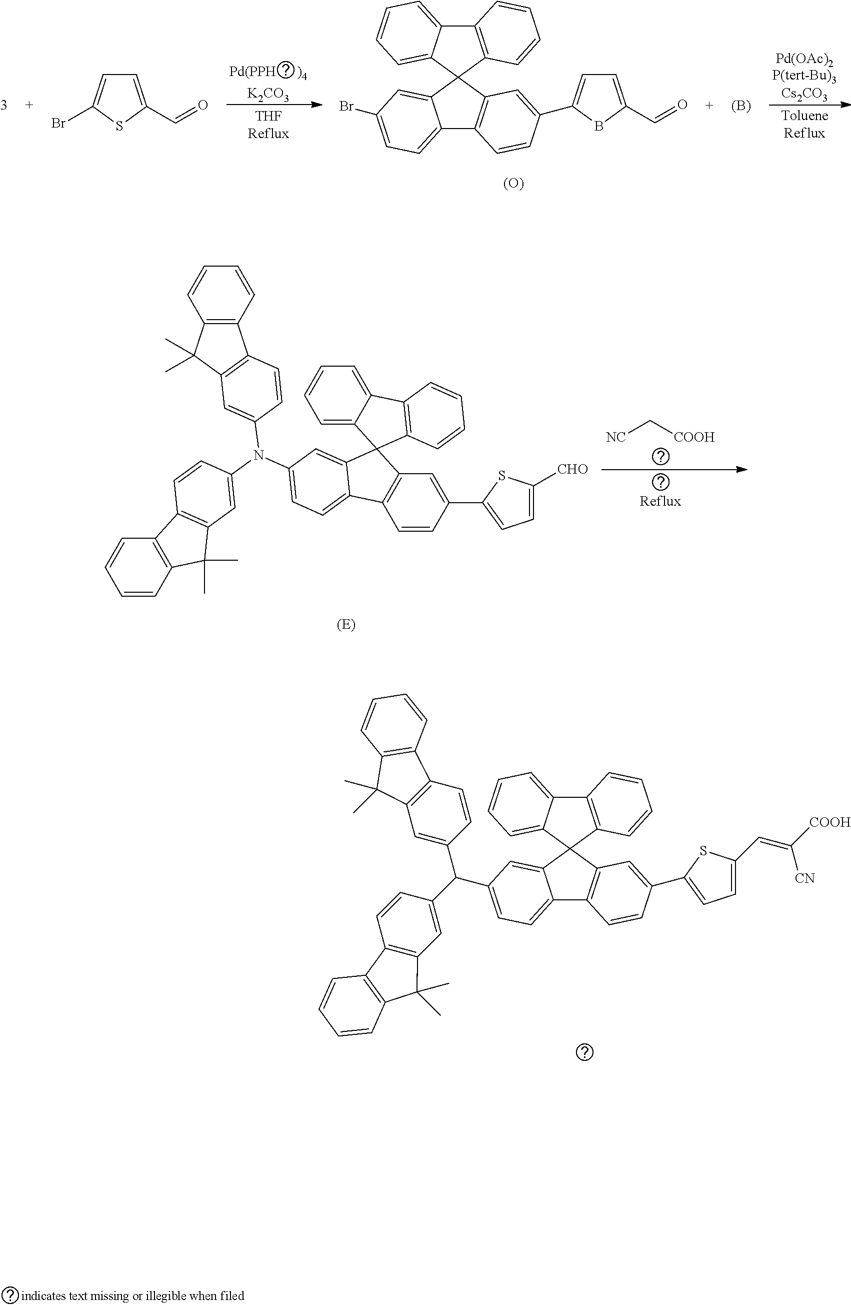

### Synthesis Example 1

### Preparation of Compound of Formula 4 (JK-87)

### Tert-Butyl bis(9,9-dimethyl-9H-fluoren-7-yl)carbamate (1)

0.014 g of copper(I) iodide (CuI(1)) (0.074 mmol), 1 g of 2-iodo-9,9-dimethylfluorene (3.12 mmol), 0.174 g of tert-butyl carbamate (1.48 mmol), and 1.45 g of Cs₂CO₃ (4.45 mmol) are added to a flask, air is removed from the flask, and the flask is then filled with a nitrogen gas. Thereafter, 0.031 ml of N,N′-dimethylethylenediamine (0.29 mmol) and 5 ml of tetrahydrofuran are added to the flask using a syringe. The flask containing the resultant mixture is refluxed at 80° C. for 36 hours. After 36 hours, the temperature of the flask is reduced to room temperature, and phase separation of the resultant mixture is performed using methylene chloride, distilled water, and a separatory funnel. Then, only a methylene chloride layer containing an organic material is separated from the resulting product, and the remaining moisture existing in the organic layer is removed with magnesium sulfate (MgSO₄). Column chromatography (stationary phase: silica gel, mobile phase:ethyl acetate:hexane=1:10 volume ratio) is performed on the resultant organic layer to obtain 0.45 g of tert-Butyl bis(9,9-dimethyl-9H-fluoren-7-yl)carbamate (1) at a yield of 60%

Mp: 156° C.

¹H NMR (300 MHz, CDCl₃): δ 7.69-7.62 (m, 4H), 7.42-7.4 (m, 2H), 7.32 (m, 6H), 7.2-7.17 (m, 2H), 1.48 (s, 12H), 1.46 (s, 9H).

¹³C NMR (300 MHz, CDCl₃): δ 154.27, 154.07, 153.9, 142.62, 138.79, 136.61, 127.13, 125.75, 122.65, 121.42, 120.06, 119.98, 81.174, 46.99, 28.44, 27.19. Anal. cal. for C₃₅H₃₅NO₂: C, 83.80; H, 7.03; N, 2.79; O, 6.38.

### Bis(9,9-dimethyl-9H-fluoren-7-yl)amine (B)

0.4 g of tert-Butoxycarbonyl-protected arylamine 1 (0.8 mmol) is dissolved in 1 ml of tetrahydrofurane, and 8 ml of trifluoroacetic acid (TFA) is added to the resulting mixture. Then, the reaction mixture is stirred at room temperature for 10 minutes, thereby obtaining a solution having a color that turns dark green. TFA is evaporated in a vacuum, dichloromethane is added to the resultant, and the resultant is neutralized using an aqueous saturated sodium hydroxide solution. The mixture is extracted with solid dichloromethane solutions as described above, and the remaining moisture in the organic layer is removed. Then, column chromatography (stationary phase:silica gel, mobile phase:ethyl acetate:hexane=1:10 volume ratio) is performed on the resultant solution to obtain 0.3 g of pale yellow Compound 2 at a yield of 95%.

Mp: 178° C.

¹H NMR (300 MHz, (CD₃)₂CO): δ 7.7-7.67 (m, 4H), 7.48 (d, J=6.9 Hz), 7.36 (s, 2H), 7.31-7.22 (m, 4H), 7.18 (d, 2H J=7.2 Hz), 1.46 (s, 12H).

¹³C NMR (300 MHz, (CD₃)₂CO): δ 155.97, 153.83, 144.45, 140.29, 132.48, 127.79, 126.73, 123.27, 121.67, 119.69, 117.32, 112.46, 47.28, 27.53.

### 7-(Bis(9,9-dimethyl-9H-fluoren-7-yl)amino)-9.9-spirobifluorene-2-carbaldehyde (2)

0.3 g of 2-bromo-7-formyl-9,9-spirobifluorene (A) (0.7 mmol), 0.44 g of Compound B (1.05 mmol), 0.023 g of palladium acetate (Pd(OAc)₂) (0.1 mmol), 0.04 g of tertiarybutylphosphine (P(tBu)₃) (0.2 mmol), and 1.4 g of cesium carbonate (Cs₂CO₃) (4.3 mmol) are dissolved in 15 ml of distilled toluene in a flask filled with a nitrogen gas, and the mixture is refluxed at 130° C. overnight. After the reaction is completed, the temperature of the flask is reduced to room temperature, and an aqueous saturated ammonium chloride solution is added to the resultant mixture. A dichloromethane layer is extracted, and the remaining moisture existing in the layer is removed with MgSO₄. Then, column chromatography (stationary phase: silica gel, mobile phase: dichloromethane:hexane=3:1 volume ratio) is performed on the resultant to obtain 0.26 g of a desired Compound 2 at a yield of 50%.

Mp: 180° C.

¹H NMR (300 MHz, (CD₃)₂CO): δ 9.85 (s, 1H), 8.12 (d, 1H. J=8.4 Hz), 8.03 (d, 1H, J=8.4 Hz), 7.97 (d, 1H, J=7.8 Hz), 7.86 (d, 2H, J=7.2 Hz), 7.7 (d, 2H, J=7.2 Hz), 7.64 (d, 2H, J==8.1 Hz), 7.45 (d, 2H, J=7.2 Hz), 7.35 (m, 12H), 6.96 (dd, 2H, J=8.1 Hz), 6.84 (d, 2H, J=7.5 Hz), 6.56 (s, 1H), 1.28 (s, 12H).

¹³CNMR (300 MHz, (CD₃)₂CO): δ 191.91, 155.95, 154.39, 152.44, 150.41, 148.7, 148.53, 147.5, 142.55, 139.48, 136.41, 135.76, 135, 131.49, 129.01, 127.9, 127.66, 124.74, 124.65, 124.49, 123.41, 123.29, 121.75, 121.25, 120.74, 120.43, 119.99, 117.87, 66.52, 54.93, 47.42, 27.56.

### 3-(2-(Bis(9,9-dimethyl-9H-fluoren-7-yl)amino)-9,9-spirobifluoren-7-yl)-2-cyanoacrylic Acid (JK-87)

0.14 g of Compound 2 (0.188 mmol) and 0.025 g of cyanoacetatic acid (0.3 mmol) are dissolved in 15 ml of distilled chloroform, and 0.025 ml of piperidine (0.29 mmol) is added to the mixture by using a syringe. Then, the resultant mixture is refluxed for 10 hours.

After the reaction is completed, the temperature of the resultant mixture is reduced to room temperature, and a 0.1M aqueous hydrogen chloride solution is added to the resultant mixture. The reaction mixture is extracted with chloroform, and the remaining moisture existing in the chloroform layer is removed with MgSO₄. Then, column chromatography (stationary phase: silica gel, mobile phase: dichloromethane:methanol=10:1 volume ratio) is performed on the resultant solution to obtain 0.068 g of the compound of Formula 4 (JK-87) at a yield of 45%.

Mp: 243° C.

¹H NMR (300 MHz, (CD₃)₂SO): δ 8.02 (m, 3H), 7.83 (d, 2H J=7.5 Hz), 7.75 (s, 1H), 7.67 (m, 4H), 7.46 (d, 2H, J=7.5 Hz), 733 (m, 6H), 7.16 (m, 4H), 7.02 (m, 2H), 6.85 (dd, 2H, J=8.1 Hz), 6.76 (d, 2H, J=7.8 Hz), 6.3 (s, 1H), 1.18 (s, 12H).

¹³C NMR (300 MHz, (CD₃)₂SO): δ 163.32, 154.76, 153.21, 150.68, 148.83, 148.28, 147.65, 147.07, 146.1, 143.82, 141.1, 138.08, 134.31, 131.87, 129.76, 128.24, 128.11, 127.14, 126.89, 124.26, 123.6, 123.48, 122.72, 122.37, 122, 121.22, 120.6, 120.25, 119.71, 119.27, 118.8, 116.21, 111.99, 110.99, 65.31, 46.41, 26.72.

### Synthesis Example 2

### Preparation of compound of Formula 5 (JK-88)

Compound 3

### {2-(2-Bromo-9,9-spirobifluoren-7-A-4,4,5,5-tetramethl-1,3,2-dioxaborolan}

3.5 g of 2,7-dibromo-9,9-spirobifluorene (7.38 mmol) is dissolved in 80 ml of tetrahydrofuran in a flask filled with a nitrogen gas, and the temperature of the flask is reduced to −78° C. 5.1 ml of normal-butyllithium (1.6M hexane solution) is added dropwise to the resultant mixture via a syringe. The resultant mixture is stirred at −78° C. for 30 minutes, and 1.7 ml of 2-isopropoxy-4,4,5,5-tetramethyl-1,3,2-dioxaboralne (8.33 mmol) is slowly added to the resultant mixture. The reaction temperature is raised to room temperature, and the resultant mixture is then stirred for 8 hours. Water is added to the resulting mixture to complete the reaction. The reaction mixture is extracted with dichloromethane, and the remaining moisture existing in the layer is removed with MgSO₄. Then, column chromatography (stationary phase: silica gel, mobile phase:ethylacetate:hexane=1:10 volume ratio) is performed on the resultant layer to obtain 3.26 g of a desired Compound 3 at a yield of 85%.

Mp: 305° C.

¹HNMR (300 MHz, CDCl₃): δ 7.85 (d, 4H J=7.2 Hz), 7.73 (d, 1H J=7.2 Hz), 7.48 (d, 1H J=7.2 Hz), 7.38 (dd, 2H J=7.2 Hz), 7.16 (s, 1H), 7.11 (dd, 2H J=7.2 Hz), 6.79 (s, 1H), 6.71 (d, 2H J=7.2 Hz), 1.25 (s, 12H).

¹³CNMR (300 MHz, CDCl₃): δ 151.8, 147.8, 147.61, 143.8, 142, 140.4, 135.08, 134.93, 131.01, 130.41, 128.08, 128.04, 127.34, 124.29, 122.11, 121.84, 120.29, 119.51, 83.9, 65.9, 48.2, 24.9.

### 4-(2-Bromo-9,9-spirobifluoren-7-yl)benzaldehyde (C)

1.52 g of Compound 3 (2.92 mmol), 0.54 g of 4-bromobenzaldehyde (2.92 mmol), 0.23 g of palladium acetate (0.2 mmol), 4.04 g of calcium carbonate (29.2 mmol), and distilled water without oxygen are dissolved in 60 ml of distilled tetrahydrofuran in a flask filled with a nitrogen gas, and the mixture is refluxed overnight. The temperature of the flask is reduced to room temperature, and the phase separation of the resultant mixture is performed using methylene chloride, distilled water, and a separatory funnel. Then, only a methylene chloride layer containing an organic material is separated from the resulting product, and the remaining moisture existing in the organic layer is removed with MgSO₄. Then, column chromatography (stationary phase: silica gel, mobile phase: dichloromethane:hexane=3:10 volume ratio) is performed on the resultant layer to obtain 1.23 g of desired Compound C at a yield of 85%.

Mp: 186° C.

¹HNMR (300 MHz, CDCl₃): δ 9.97 (s, 1H), 7.93 (m, 3H), 7.83 (d, 2H, J=8.4 Hz), 7.77 (d, 1H, ³J=8.7), 7.69 (dd, 1H, J=8.4 Hz), 7.58 (d, 2H, J=8.7 Hz), 7.54 (dd, 1H, J=8.1 Hz), 7.44 (m, 2H), 7.18 (m, 2H), 7.1 (s, 1H), 6.89 (s, 1H), 6.8 (d, 2H, J=7.8 Hz).

¹³C NMR (300 MHz, CDCl₃): δ 191.87, 151.41, 149.67, 147.72, 146.76, 141.9, 141.25, 140.07, 139.87, 135.21, 131.24, 130.2, 128.31, 127.68, 124.22, 123, 122.09, 121.71, 120.73, 120.4, 66, 48.2.

### 4-(2-(Bis(9,9-dimethyl-9H-fluoren-7-yl)amino)-9,9-spirobifluoren-7-yl)benzaldehyde

0.5 g of Compound B (1.25 mmol), 0.4 g of Compound C (0.8 mmol), 0.023 g of palladium acetate (Pd(OAc)₂) (0.1 mmol), 0.04 g of t-ebutylphosphine (P(tBu)₃) (0.2 mmol), and 1.4 g of cesium carbonate (Cs₂CO₃) (4.3 mmol) are dissolved in 15 ml of distilled toluene, and the mixture is refluxed at 130° C. overnight.

After the reaction is completed, the temperature of the flask is reduced to room temperature, and an aqueous saturated ammonium chloride solution is added to the resultant mixture. A dichloromethane layer is extracted, and the remaining moisture existing in the layer is removed with MgSO₄. Then, column chromatography (stationary phase: silica gel, mobile phase: dichloromethane:hexane=3:1 volume ratio) is performed on the resultant to obtain 0.33 g of a desired Compound 4 at a yield of 50%.

Mp: 185° C.

¹H NMR (300 MHz, CDCl₃): δ 9.97 (s, 1H), 7.9 (m, 6H), 7.69 (m, 8H), 7.41 (m, 9H), 7.2 (m, 4H), 7.03 (m, 4H), 6.75 (s, 1H), 1.33 (s, 12H).

¹³C NMR (300 MHz, CDCl₃): δ 191.85, 155.03, 153.6, 150.7, 149.96, 148.59, 148.41, 147.04, 142.26, 141.8, 138.97, 138.29, 135.42, 134.95, 134.35, 130.15, 128.03, 127.33, 127.07, 126.6, 124.29, 123.45, 122.7, 122.54, 121.05, 120.63, 120.29, 119.87, 119.5, 118.65, 66.09, 46.83, 27.13.

### 3-(4-(2-(Bis(9,9-dimethyl-9H-fluoren-7-yl)amino)-9,9-spirobifluoren-7-yl)phenyl)-2-cyanoacrylic Acid (JK-88)

0.27 g of Compound 4 (0.33 mmol) and 0.056 g of cyanoacetic acid (0.66 mmol) are dissolved in 8 ml of distilled chloroform, 0.065 ml of piperidine (0.66 mmol) is added to the mixture via a syringe, and the resultant mixture is then refluxed for 10 hours.

After the reaction is completed, the temperature of the resultant mixture is reduced to room temperature, and a 0.1M aqueous hydrogen chloride solution is added to the resultant mixture. A chloroform layer is extracted, and the remaining moisture existing in the layer is removed with MgSO₄. Then, column chromatography (stationary phase: silica gel, mobile phase: dichloromethane:methanol=10:1 volume ratio) is performed on the resultant to obtain 0.32 g of the compound JK-88 at a yield of 55%.

Mp: 263° C.

¹H NMR (300 MHz, (CD₃)₂SO): δ 7.99 (m, 3H), 7.82 (m, 4H), 7.65 (d, 2H, J=6.9 Hz), 7.59 (d, 2H, J=8.1 Hz), 7.51 (d, 2H, J=7.2 Hz), 7.43 (d, 2H, J=6.3 Hz), 7.28 (m, 9H), 7.12 (m, 4H), 6.82 (m, 4H), 6.31 (s, 1H), 1.15 (s, 12H).

¹³C NMR (300 MHz, (CD₃)₂SO): δ 163.69, 154.77, 153.21, 150.29, 149.27, 148.06, 147.75, 147.4, 146.3, 141.89, 141.42, 141.16, 138.15, 137.73, 134.93, 134.15, 131.97, 130.17, 128.27, 128.11, 127.18, 126.84, 123.56, 123.42, 122.75, 122.34, 121.93, 121.22, 120.63, 119.71, 119.03, 118.61, 116.75, 112.43, 46.42, 26.75.

### Synthesis Example 3

### Preparation of Compound of Formula 6 (JK-89)

### 5-(2-Bromo-9,9-spirobifluoren-7-yl)thiophene-2-carbaldehyde (D)

0.45 g of Compound 3 (0.86 mmol), 0.15 ml of 5-bromothiophene-2-carbaldehyde (1.3 mmol), 0.07 g of palladium acetate (0.06 mmol), 1.19 g of potassium carbonate (8.61 mmol), and 4.3 ml of distilled water without oxygen are dissolved in tetrahydrofurane, and the mixture is refluxed overnight to obtain 0.34 g of Compound D at a yield of 80%.

Mp: 225° C.

¹H NMR (300 MHz, CDCl₃): δ 9.79 (s, 1H), 7.89 (m, 3H), 7.74 (dd, 2H), 7.6 (d, 1H, J=4.8 Hz), 7.53 (dd, 1H), 7.42 (m, 2H), 7.19 (d, 1H, J=4.2 Hz), 7.14 (m, 2H), 6.99 (dd, 1H), 6.86 (s, 1H), 6.76 (dd, 2H).

¹³C NMR (300 MHz, CDCl₃): δ 182.64, 153.8, 151.31, 149.74, 148.13, 147.31, 142.23, 142.02, 141.79, 139.73, 137.29, 136.65, 134.68, 133.04, 131.52, 131.25, 130.48, 128.36, 127.38, 126.66, 124.07, 122.3, 121.8, 121.79, 120.87, 120.4, 83.29, 48.24.

### 5-(2-(Bis(9,9-dimethyl-9H-fluoren-7-yl)amino)-9,9-spirobifluoren-7-yl)thiophene-2-carbaldehyde (E)

1.05 g of Compound B (2.61 mmol), 0.88 g of Compound D (1.74 mmol), 0.016 g of palladium acetate (Pd(OAc)₂) (0.071 mmol), 0.03 g of t-cbutylphosphine (P(tBu)₃) (0.148 mmol), and 1.25 g of cesium carbonate (Cs₂CO₃) (3.84 mmol) are dissolved in 30 ml of distilled toluene, and the mixture is refluxed at 130° C. overnight.

After the reaction is completed, the temperature of the flask is reduced to room temperature, and an aqueous saturated ammonium chloride solution is added to the resultant mixture. The resultant mixture is extracted with chloroform and the remaining moisture existing in the layer is removed with MgSO₄. Then, column chromatography (stationary phase: silica gel, mobile phase: dichloromethane:hexane=3:1 volume ratio) is performed on the resultant to obtain 1.15 g of a desired Compound E at a yield of 60%.

Mp: 197° C.

¹H NMR (300 MHz, CDCl₃): δ 9.96 (s, 1H), 7.98 (m, 6H), 7.77 (dd, 3H), 7.68 (d, 2H, J=7.5 Hz), 7.55 (m, 8H), 7.31 (dd, 6H), 7.11 (m, 4H), 6.85 (s, 1H), 1.46 (s, 12H).

¹³C NMR (300 MHz, CDCl₃): δ 182.69, 155.06, 154.64, 154.64, 150.74, 150.12, 148.28, 146.97, 143.22, 141.89, 141.8, 138.97, 137.35, 135.08, 134.46, 131.63, 128.07, 127.08, 126.64, 124.16, 124.01, 123.91, 123.55, 123.28, 122.57, 121.68, 121.14, 120.65, 120.36, 119.97, 119.52, 118.74, 118.47, 65.99, 46.86, 27.14.

### 3-(5-(2-(Bis(9,9-dimethyl-9H-fluoren-7-yl)amino)-9,9-spirobifluoren-7-yl)thiophen-2-yl)-2-cyanoacrylic Acid (JK-89)

0.19 g of Compound E (0.23 mmol) and 0.04 g of cyanoacetate (0.43 mmol) are dissolved in 12 ml of distilled chloroform, 0.047 ml of piperidine (0.47 mmol) is added to the mixture via a syringe, and the resultant mixture is then refluxed for 10 hours.

After the reaction is completed, the temperature of the resultant mixture is reduced to room temperature, and a 0.1M aqueous hydrogen chloride solution is added to the resultant mixture. The reaction mixture is extracted with chloroform, and the remaining moisture existing in the layer is removed with MgSO₄. Then, column chromatography (stationary phase: silica gel, mobile phase: dichloromethane:methanol=10:1 volume ratio) is performed on the resultant to obtain 0.1 g of the compound JK-89 at a yield of 50%.

Mp: 285° C.

¹H NMR (300 MHz, (CD₃)₂SO): δ 8.06 (s, 1H), 7.98 (m, 2H), 7.86 (d, 2H, J=7.2 Hz), 7.79 (d, 1H, J=8.1 Hz), 7.67 (d, 2H, J=7.2 Hz), 7.62 (d, 2H, J=8.4 Hz), 7.45 (m, 3H), 7.34 (d, 1H, J=7.8 Hz), 7.28 (m, 7H), 7.14 (m, 4H), 7.04 (d, 1H, J=8.4 Hz), 6.84 (m, 4H), 6.3 (s, 1H), 1.18 (s, 12H).

¹³C NMR (300 MHz, (CD₃)₂SO): δ 189.11, 163.51, 154.67, 153.12, 150.24, 149.19, 148.46, 147.7, 147.84, 146.13, 142.08, 141.29, 141.01, 138.04, 135.62, 134.43, 134.11, 131.41, 128.24, 128.11, 127.04, 126.76, 126.24, 124.48, 123.47, 123.36, 122.62, 122.2, 121.92, 121.1, 120.67, 119.91, 119.91, 119.6, 118.57, 118.82, 65.34, 46.33, 26.64.

### Example 1

### Manufacture of Dye-Sensitized Solar Cell

A dispersion of titanium oxide particles each having a diameter of about 10 nm was coated onto a 1 cm² area of a conductive film, formed of ITO, of a first electrode by using a doctor blade. The resultant layer was heat-treated and sintered at 450° C. for 30 minutes to prepare a porous film having a thickness of 10 p.m.

Subsequently, the temperature of the resultant film was maintained at 80° C., and then the resultant film was impregnated in 0.3 mM of a dye dispersion in which the compound of Formula 4 was dissolved in ethanol, and a dye adsorption treatment was performed for 12 hours or more.

The dye-adsorbed porous film was cleaned using ethanol and dried at room temperature to manufacture the first electrode including a light absorption layer.

Separately, a platinum catalyst electrode was formed on the conductive film formed of ITO from above to form a second electrode. In order to facilitate injection of an electrolyte, fine holes were formed using a drill having a diameter of 0.75 mm.

A support that was formed of a thermoplastic polymer film (Surlyn, DuPont, USA) and having a thickness of 60 μM. It was positioned between the first electrode with the porous film formed thereon and the second electrode. Then, the resultant multi-layer structure was pressed under pressure at 100° C. for 9 seconds to join the first and second electrodes together. Then, the electrolyte was injected into the interior between the first electrode and the second electrode through the fine holes formed in the second electrode. The fine holes were sealed using a cover glass and a thermoplastic polymer film to complete the manufacture of the dye-sensitized solar cell.

The electrolyte used was prepared by dissolving 0.6 M of 1,2-dimethyl-3-hexylimidazolium iodide, 0.5 M of 4-tert-butylpyrimidine, 0.1M of Li1, and 0.05 M of I₂ in acetonitrile.

### Example 2

### Manufacture of Dye-Sensitized Solar Cell

A dye-sensitized solar cell was manufactured in the same manner as in Example 1, except that the compound of Formula 5 was used instead of the compound of Formula 4.

### Example 3

### Manufacture of Dye-Sensitized Solar Cell

A dye-sensitized solar cell was manufactured in the same manner as in Example 1, except that the compound of Formula 6 was used instead of the compound of Formula 4.

### Comparative Example 1

### Manufacture of Dye-Sensitized Solar Cell

A dye-sensitized solar cell was manufactured in the same manner as in Example 1, except that a N719 dye (Ruthenium complex dye) represented by the following formula was used instead of the compound of Formula 4:

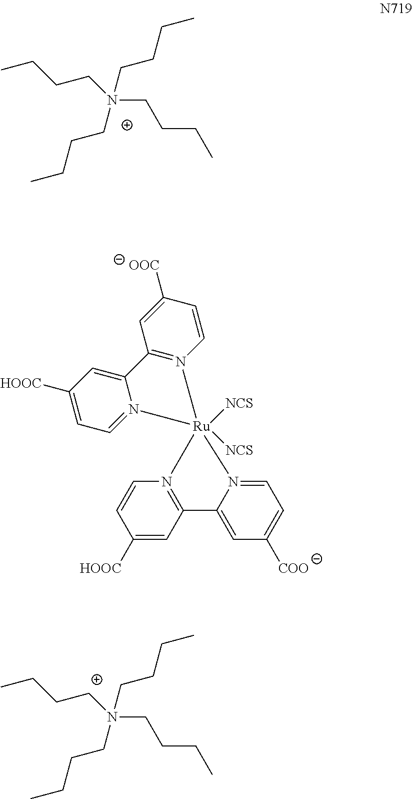

The thermal stability of each of the compounds of Formulae 4 through 6 prepared according to Synthesis Examples 1, 2, and 3, respectively was evaluated by measuring a melting point of each compound.

As a result of the evaluation, it is confirmed that each compound has excellent thermal stability.

In addition, cyclovoltammetry of each of the compounds of Formulae 4 through 6 respectively prepared, according to Examples 1 through 3 was measured, and the results are shown in Table 1 below. In the Table 1, JK-87, JK-88, and JK-88 denote the compounds of Formulae 4 through 6, respectively.

TABLE 1

| Dye | E_{redox}(ΔEₚ)N | E₀₋₀N | E_{LUMO}N |
|---|---|---|---|
| JK-87 | 1.14 | 2.47 | −1.33 |
| JK-88 | 1.12 | 2.47 | −1.36 |
| JK-89 | 1.13 | 2.43 | −1.31 |

From the results shown in Table 1, it can be seen that lowest unoccupied molecular orbital (LUMO) potentials (LUMO energy level) of the compounds of Synthesis Examples 1 through 3 are lower (i.e., −1.33 to −1.36V (NHE basis)) than the potential of a conduction band (−0.5 V) of TiO₂, and thus the compounds of Synthesis Examples 1 through 3 had bands that facilitate electron injection.

In addition, highest unoccupied molecular orbital (HOMO) potentials of the spirobifluorene-based compounds of Synthesis Examples 1 through 3 are from about 1.12 to about 1.14 V (NHE basis), which are more positive values than a redox potential of I-/I₃-(i.e., 0.4 V), and thus, it is confirmed that the compounds of Synthesis Examples 1 through 3 had bands that facilitate electron regeneration.

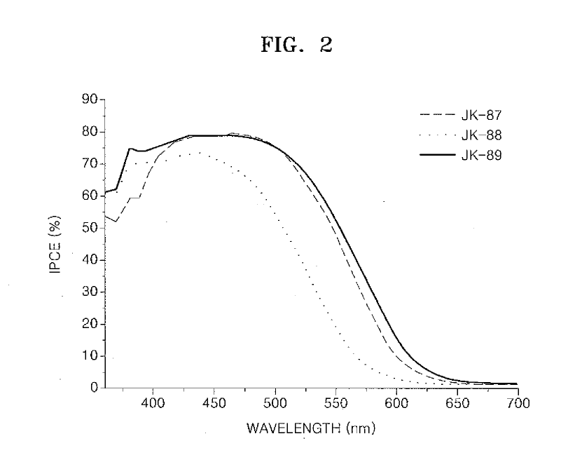

FIG. 2 is a graph showing variation in incident photon to current efficiency (IPCE) with respect to unit wavelength of the dye-sensitized solar cells manufactured according to Examples 1 through 3. In FIG. 2, JK-87, JK-88, and JK-89 are the compounds respectively used in the dye-sensitized solar cells of Examples 1, 2, and 3.

Referring to FIG. 2, the dye-sensitized solar cells of Examples 1 through 3 absorb visible rays with a wavelength of 600 nm or greater, thereby being capable of producing electricity. In addition, each dye-sensitized solar cell maintains a photoelectric conversion efficiency of 70% or greater at a wavelength from about 360 to about 540 nm, and it is confirmed that each dye-sensitized solar cell exhibits high IPCE, which is close to about 90% while taking into consideration the reflection and absorption of glass.

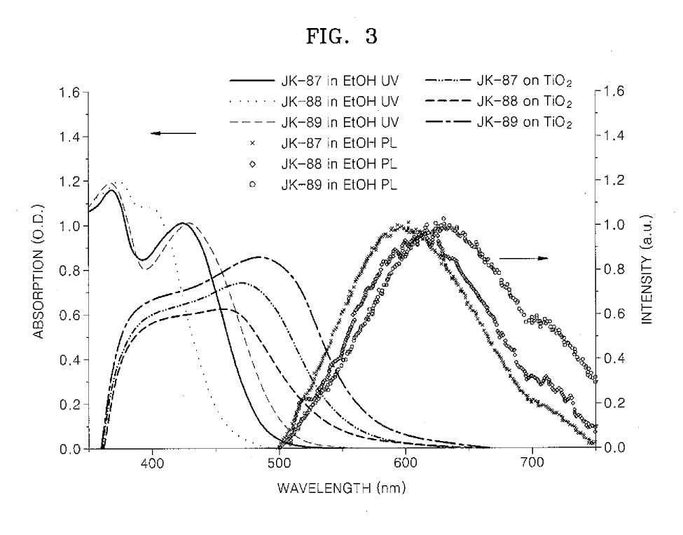

UV/photoluminescence (UV/PL) characteristics of the compounds of Formulae 4 through 6 of Synthesis Examples 1 through 3 were evaluated, and the results are shown in FIG. 3 and Table 2 below. In FIG. 3, “in EtOH” represents a case where the UV/PL characteristics are measured in an ethanol solution state, the concentration of a dye dissolved in ethanol is 3×10⁻⁵ M, and “on TiO₂” represents a UV spectra of TiO₂ films onto which one selected from the group consisting of the compounds of Formulae 4 through 6 as a dye are adsorbed.

TABLE 2

| Dye | λ_{abs}/nm (ε/M⁻¹cm⁻¹) |
|---|---|
| JK-87 | 369 (22,000) |
| | 427 (19,000) |
| JK-88 | 371 (32,000) |
| | 410 (28,000) |
| JK-89 | 368 (40,000) |
| | 428 (34,000) |

Referring to Table 2, JK-87, JK-88, and JK-89 have maximum absorption coefficients at an absorption wavelength from about 410 to 420 nm, and it is confirmed that each dye has an absorption band up to a wavelength of 500 nm. In addition, it is confirmed that the absorption coefficient of each dye is higher than that of the N719 dye (ruthenium complex dye) of Comparative Example 1 (absorption coefficient (ε): 13,000). Referring to FIG. 3, it is confirmed that the absorption wavelength after each dye is adsorbed onto the TiO₂ film is around 600 nm.

Open-circuit voltage (V_{OC}), current density (Jsc), energy conversion efficiency (E_{ff}), and fill factor (FF) of each of the dye-sensitized solar cells of Examples 1 through 3 were measured, and the results are shown in Table 3 below.

The measurement conditions of the open-circuit voltage (V_{OC}), current density (Jsc), energy conversion efficiency (E_{ff}), and fill factor (FF) in Table 3 below are as follows:

(1) Open-Circuit Voltage (V_{OC}) and Current Density (Jsc)

: The open-circuit voltage and the current density were measured using a Keithley SMU2400.

(2) Energy Conversion Efficiency (E_{ff}) and Fill Factor (FF)

The energy conversion efficiency was measured using a 1.5 AM 100 mW/cm² solar simulator (composed of an Xe lamp [300 W, Oriel], AM1.5 filter, and Keithley SMU2400), and the fill factor was calculated using the obtained energy conversion efficiency given by an Equation below:

fill factor (%)={(J×V)ₘₐₓ/(J_{sc}×V_{OC}}×100 Equation

In the above Equation, J denotes a Y-axis value of an energy conversion efficiency curve, V denotes an X-axis value of the energy conversion efficiency curve, and J_{sc} and V_{oc} denotes intercept values of each axis.

TABLE 3

| Dye | J_{SC}(mAcm⁻²) | V_{OC}(V) | FF | η(%) |
|---|---|---|---|---|
| JK-87 | 11.31 | 0.78 | 0.75 | 6.58 |
| JK-88 | 9.28 | 0.76 | 0.75 | 5.28 |
| JK-89 | 13.02 | 0.75 | 0.70 | 6.83 |

From the results shown in Table 3, it is confirmed that the dye-sensitized solar cells of Examples 1 through 3 have a higher open-circuit voltage than the open-circuit voltage (0.6 to 0.7 V) of the dye-sensitized solar cell manufactured using the conventional organic dye, and have an enhanced energy conversion efficiency and fill factor.

As described above, according to one or more of the above embodiments, a dye-sensitized solar cell manufactured using a spirobifluorene-based compound having excellent thermal stability may have an enhanced photoelectric conversion efficiency.

It should be understood that the exemplary embodiments described herein should be considered in a descriptive sense only and not for purposes of limitation. Descriptions of features or aspects within each embodiment should typically be considered as available for other similar features or aspects in other embodiments.

What is claimed is:

1. A spirobifluorene-based compound represented by Formula 1 below: 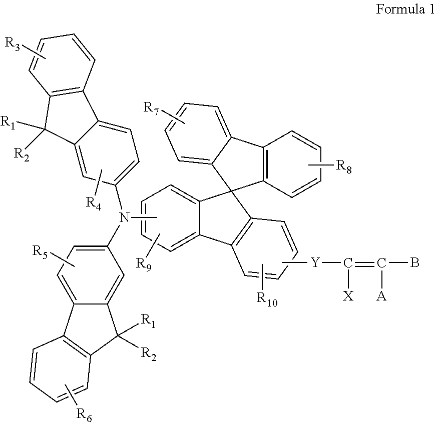
wherein Y is a chemical bond, a substituted or unsubstituted C₆-C₃₀ arylene group, a substituted or unsubstituted C₂-C₃₀ heteroarylene group, or a substituted or unsubstituted C₂-C₃₀ alkynylene group,
X is hydrogen, a substituted or unsubstituted C₁-C₃₀ alkyl group, a substituted or unsubstituted C₁-C₃₀ alkoxy group, a substituted or unsubstituted C₆-C₃₀ aryl group, a substituted or unsubstituted C₂-C₃₀ heteroaryl group, or a cyano group,
R₁ and R₂ are each independently hydrogen, a substituted or unsubstituted C₁-C₃₀ alkyl group, a substituted or unsubstituted C₁-C₃₀ alkoxy group, a substituted or unsubstituted C₆-C₃₀ aryl group, or a substituted or unsubstituted C₂-C₃₀ heteroaryl group,
A is a cyano group or a carboxyl group,
B is an acidic functional group, and
R₃ through R¹⁰ are each independently hydrogen, a substituted or unsubstituted C₁-C₃₀ alkyl group, a substituted or unsubstituted C₁-C₃₀ alkoxy group, a substituted or unsubstituted C₆-C₃₀ aryl group, a substituted or unsubstituted C₂-C₃₀ heteroaryl group, a substituted or unsubstituted C₂-C₃₀ alkynyl group, a substituted or unsubstituted C₃-C₃₀ carbocyclic group, a substituted or unsubstituted C₂-C₃₀ heterocyclic group, a halogen atom, a hydroxyl group, a cyano group, a thiol group, or an amino group.

2. The spirobifluorene-based compound of claim 1, wherein Y is a single bond, a C₆-C₂₀ arylene group, or a C₂-C₂₀ heteroarylene group.

3. The spirobifluorene-based compound of claim 1, wherein B is at least one selected from the group consisting of a carboxyl group, a phosphorous group, a sulfonic acid group, a phosphinic acid group, an oxycarboxylic acid group, a boric acid group, and a squaric acid group.

4. The spirobifluorene-based compound of claim 1, wherein the spirobifluorene-based compound is the compound represented by Formula 2 below: 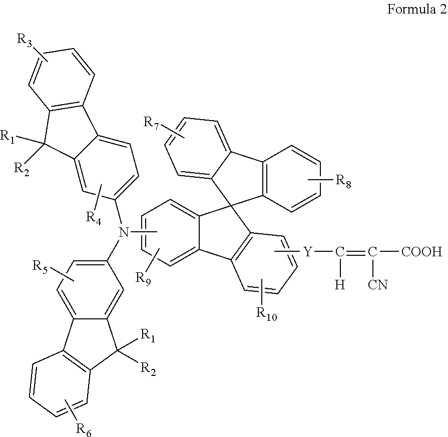
wherein Y denotes a chemical bond, or a substituted or unsubstituted C₆-C₃₀ arylene group, a substituted or unsubstituted C₂-C₃₀ heteroarylene group, or a substituted or unsubstituted C₂-C₃₀ alkynylene group,
R₁ and R₂ are each independently hydrogen, a substituted or unsubstituted C₁-C₃₀ alkyl group, a substituted or unsubstituted C₁-C₃₀ alkoxy group, a substituted or unsubstituted C₆-C₃₀ aryl group, or a substituted or unsubstituted C₂-C₃₀ heteroaryl group, and
R₃ through R₁₀ are each independently hydrogen, a substituted or unsubstituted C₁-C₃₀ alkyl group, a substituted or unsubstituted C₁-C₃₀ alkoxy group, a substituted or unsubstituted C₆-C₃₀ aryl group, a substituted or unsubstituted C₂-C₃₀ heteroaryl group, a substituted or unsubstituted C₂-C₃₀ alkynyl group, a substituted or unsubstituted C₃-C₃₀ carbocyclic group, a substituted or unsubstituted C₂-C₃₀ heterocyclic group, a halogen atom, a hydroxyl group, a cyano group, a thiol group, or an amino group.

5. The spirobifluorene-based compound of claim 1, wherein the spirobifluorene-based compound is the compound represented by Formula 3 below: 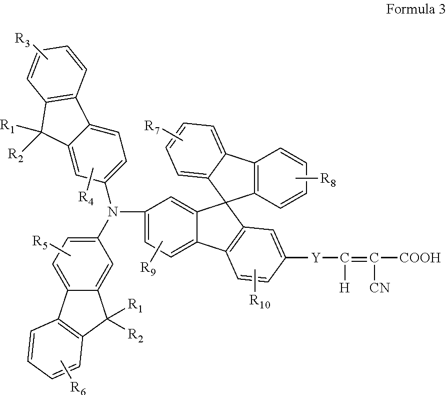
wherein Y denotes a chemical bond, or a substituted or unsubstituted C₆-C₃₀ arylene group, a substituted or unsubstituted C₂-C₃₀ heteroarylene group, or a substituted or unsubstituted C₂-C₃₀ alkynylene group,
R₁ and R₂ are each independently hydrogen, a substituted or unsubstituted C₁-C₃₀ alkyl group, a substituted or unsubstituted C₁-C₃₀ alkoxy group, a substituted or unsubstituted C₆-C₃₀ aryl group, or a substituted or unsubstituted C₂-C₃₀ heteroaryl group, and
R₃ through R₁₀ are each independently hydrogen, a substituted or unsubstituted C₁-C₃₀ alkyl group, a substituted or unsubstituted C₁-C₃₀ alkoxy group, a substituted or unsubstituted C₆-C₃₀ aryl group, a substituted or unsubstituted C₂-C₃₀ heteroaryl group, a substituted or unsubstituted C₂-C₃₀ alkynyl group, a substituted or unsubstituted C₃-C₃₀ carbocyclic group, a substituted or unsubstituted C₂-C₃₀ heterocyclic group, a halogen atom, a hydroxyl group, a cyano group, a thiol group, or an amino group.

6. The spirobifluorene-based compound of claim 5, wherein Y is a single bond, a C₆-C₂₀ arylene group, or a C₂-C₂₀ heteroarylene group.

7. The spirobifluorene-based compound of claim 5, wherein Y is one selected from the groups represented by the following formulae:

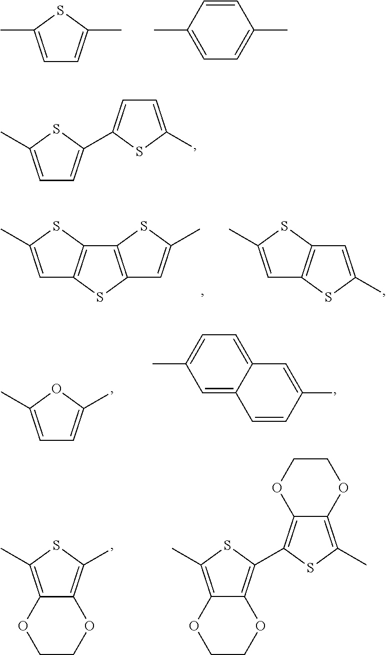

8. The spirobifluorene-based compound of claim 1, wherein the spirobifluorene-based compound is one selected from compounds represented by Formulae 4 through 6 below:

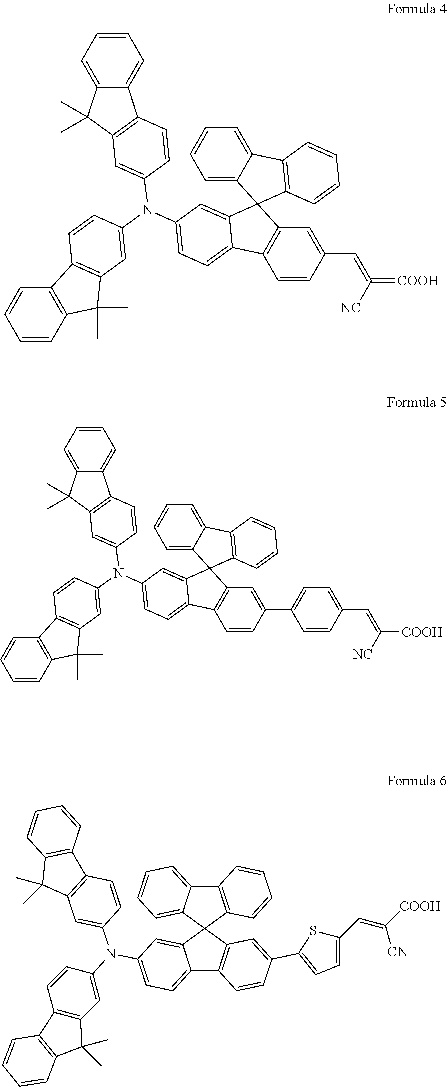

9. The spirobifluorene-based compound of claim 1, wherein the spirobifluorene-based compound is the compound represented by Formula 4 below:

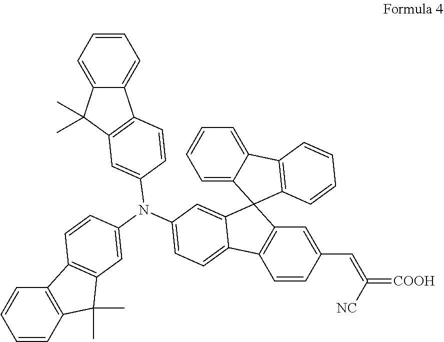

10. The spirobifluorene-based compound of claim 1, wherein the spirobifluorene-based compound is the compound represented by Formula 5 below:

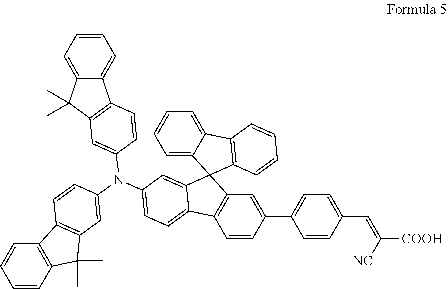

11. A dye-sensitized solar cell comprising:
a first electrode, a light absorption layer formed on a surface of the first electrode, a second electrode disposed to face the first electrode on which the light absorption layer is formed, and an electrolyte disposed between the first electrode and the second electrode; and
a spirobifluorene-based compound according to claim 1, wherein the spirobifluorene-based compound is used as a dye.

12. A dye-sensitized solar cell comprising:
a first electrode, a light absorption layer formed on a surface of the first electrode, a second electrode disposed to face the first electrode on which the light absorption layer is formed, and an electrolyte disposed between the first electrode and the second electrode; and
a spirobifluorene-based compound according to claim 1, wherein the spirobifluorene-based compound is used as a dye.

13. A dye-sensitized solar cell comprising:
a first electrode, a light absorption layer formed on a surface of the first electrode, a second electrode disposed to face the first electrode on which the light absorption layer is formed, and an electrolyte disposed between the first electrode and the second electrode; and
a spirobifluorene-based compound according to claim 2, wherein the spirobifluorene-based compound is used as a dye.

14. A dye-sensitized solar cell comprising:
a first electrode, a light absorption layer formed on a surface of the first electrode, a second electrode disposed to face the first electrode on which the light absorption layer is formed, and an electrolyte disposed between the first electrode and the second electrode; and
a spirobifluorene-based compound according to claim 3, wherein the spirobifluorene-based compound is used as a dye.

15. A dye-sensitized solar cell comprising:
a first electrode, a light absorption layer formed on a surface of the first electrode, a second electrode disposed to face the first electrode on which the light absorption layer is formed, and an electrolyte disposed between the first electrode and the second electrode; and
a spirobifluorene-based compound according to claim 4, wherein the spirobifluorene-based compound is used as a dye.

16. A dye-sensitized solar cell comprising:
a first electrode, a light absorption layer formed on a surface of the first electrode, a second electrode disposed to face the first electrode on which the light absorption layer is formed, and an electrolyte disposed between the first electrode and the second electrode; and
a spirobifluorene-based compound according to claim 5, wherein the spirobifluorene-based compound is used as a dye.

17. A dye-sensitized solar cell comprising:
a first electrode, a light absorption layer formed on a surface of the first electrode, a second electrode disposed to face the first electrode on which the light absorption layer is formed, and an electrolyte disposed between the first electrode and the second electrode; and
a spirobifluorene-based compound according to claim 6, wherein the spirobifluorene-based compound is used as a dye.

18. A dye-sensitized solar cell comprising:
a first electrode, a light absorption layer formed on a surface of the first electrode, a second electrode disposed to face the first electrode on which the light absorption layer is formed, and an electrolyte disposed between the first electrode and the second electrode; and
a spirobifluorene-based compound according to claim 7, wherein the spirobifluorene-based compound is used as a dye.

19. A dye-sensitized solar cell comprising:
a first electrode, a light absorption layer formed on a surface of the first electrode, a second electrode disposed to face the first electrode on which the light absorption layer is formed, and an electrolyte disposed between the first electrode and the second electrode; and
a spirobifluorene-based compound according to claim 8, wherein the spirobifluorene-based compound is used as a dye.

20. A dye-sensitized solar cell comprising:
a first electrode, a light absorption layer formed on a surface of the first electrode, a second electrode disposed to face the first electrode on which the light absorption layer is formed, and an electrolyte disposed between the first electrode and the second electrode; and
a spirobifluorene-based compound according to claim 9, wherein the spirobifluorene-based compound is used as a dye.
